(19) United States
(12) Patent Application Publication
(10) Pub. No.: US 2024/0060945 A1
(43) Pub. Date: Feb. 22, 2024

(54) Apparatus For Controlling Water Consumption In Bathrooms

(71) Applicant: AGUARDIO APS, Vejle (DK)

(72) Inventors: Thomas MUNCH-LAURSEN, Kolding (DK); Kristian Reesen SKOUBOE, Flensburg (DE)

(73) Assignee: AGUARDIO APS, Vejle (DK)

(21) Appl. No.: 18/267,008

(22) PCT Filed: Dec. 16, 2021
(86) PCT No.: PCT/DK2021/050370

§ 371 Date: Jun. 13, 2023

(30) Foreign Application Priority Data
Dec. 18, 2020 (DK) PA 2020 70854

(51) Int. Cl. G01N 33/00; G01P 13/00; G01D 5/14; G01K 3/10; G05B 19/416

(52) U.S. Cl. CPC G01N 33/0027; G01P 13/00; G01D 5/145; G01K 3/10; G05B 19/416; G06K 19/06037

(57) ABSTRACT

An apparatus and system for reducing or controlling water consumption in bathrooms includes a housing and is configured to receive data from water consumption locations. The housing includes a motion sensor to detect movement in a threshold distance from the apparatus as a first indication of activity, an audio sensor to monitor sound of running water as a second indication of activity, optionally a humidity sensor to monitor ambient humidity as a third indication of activity, optionally an ambient temperature sensor, a controller to qualify data received from the sensors by an algorithm, a transmitter to transmit data to an external unit, optionally via a mobile device, by low power signals, and an identification mechanism readable or scannable by a mobile device and configured to include output data based on qualified data for reducing or controlling water consumption.

### FIELD OF THE INVENTION

The present invention relates to the field of devices and systems for reducing or controlling water consumption in bathrooms. In particular, the invention relies on intelligent use of sensor technology for detecting human activity in bathrooms.

### BACKGROUND OF THE INVENTION

Water consumption and excessive use of fresh water in bathrooms have been subject to attention for decades. Fresh water shortage and increased wastewater emissions are some of the concerns that become more and more relevant as human water consumption is rapidly increasing. Usually, showers are supplied by fresh drinking water which is generally of public concern in terms of maintaining a sustainable supply of drinking water.

Additionally, showers are one of the single most water consuming installations in bathrooms, and the water often requires heavy energy consumption due to increased temperature of the water used. In terms of obtaining a sustainable level of CO2 emission, it is generally a desire to limit human activity in showers and other water consuming locations in bathrooms. Reducing water consumption in showers by minutes or even seconds would generally accomplish a milestone in energy consumption balance budgets.

Further problems with respect to water consumption arise for installations in bathrooms over time. Old installations or maintenance deficiencies often give rise to excessive use of fresh water in bathrooms. This may for example occur for running toilets where fresh drinking water may be constantly supplied and washed out due to problems with faulty flappers or broken valves. Likewise, water taps or showers may be subject to running water due to maintenance issues, but also if the installations are not closed properly or even left running after use. A fatal consequence may be floating due to running water which may have huge economic consequences.

Accordingly, solutions to the problems mentioned above are highly desired and would contribute with a positive carbon footprint as well as more controlled water consumption in bathrooms. While solutions have been provided to reduce water consumption for individual installations in the prior art, for instance by applying a timer to a shower, these solutions have drawbacks since they do not allow for flexibility, human interaction, and human awareness.

Consolidated reports have shown that human awareness is important in order to address the problem of water consumption in bathrooms. Hence, there is a need for solutions that both contribute to limiting water consumption in bathrooms and at the same time provide human awareness that serves improved water utilization in the long run. Behavioral interaction data intelligently collected and further processed may contribute to increased awareness and sophisticated solutions for reducing and controlling water consumption in bathrooms.

Additionally, there is a need for solutions that in some embodiments incorporate more than a single water installation in bathrooms, contributing to a coordinated effort in controlling more than one water installation in a bathroom, such as all installations in a bathroom at the same time. Coordinated data collection from more than one water installation in a bathroom may serve to accomplish intelligent use of water installations and may contribute to identifying derived symptoms among user of the bathroom, including diseases such as incontinence and cystitis.

Finally, there is a need for solutions that can detect running water and provide alerts when needed under certain conditions, as well as solutions that may help nudge humans to use bathroom facilities in the most proper way, including coordinated use of water installations for hygiene purposes. These needs and problems in addition to further problems may be solved according to the present invention described in the following description and outlined in detail in the accompanying drawings

### SUMMARY OF THE INVENTION

Accordingly, there is provided an apparatus (100) for reducing or controlling water consumption in bathrooms, the apparatus (100) comprising a housing (105) and being configured to receive data from one or more water consumption locations in a bathroom, the housing (105) comprising:
one or more sensors (107) including a motion sensor (110) configured to detect a movement in a certain distance from the apparatus in a bathroom as a first indication of activity in the bathroom, an audio sensor (115) configured to monitor a sound of running water from one or more water consumption locations in a bathroom as a second indication of activity in the bathroom, optionally a humidity sensor (120) configured to monitor the ambient humidity in a bathroom as a third indication of activity in the bathroom, and optionally an ambient temperature sensor (200);
a controller (125) configured to qualify at least a part of the data received from the one or more sensors (107) by means of an algorithm to obtain qualified data;
a transmitting member (130) configured to transmit the qualified data at a given point of time to an external unit (135), optionally via a mobile device (230), by means of low power signals; and
a unique identification means (140) being readable or scannable by a mobile device and configured to include output data based on the qualified data for reducing or controlling water consumption in the bathroom.

Additionally, in a second aspect, there is provided a system (201) for reducing or controlling water consumption in bathrooms, the system comprising:
an apparatus (100) according to any one of the preceding claims;
an external unit (135) configured to receive qualified data from the apparatus (100) at a given point of time, optionally via a mobile device (230), by means of low power signals, and being configured to process data based on the qualified data and/or based on further qualification of data, such as humidity data, and configured to incorporate output data in a unique identification means (140) being readable by a mobile device (230) on the apparatus (100) for reducing or controlling water consumption in a bathroom;
optionally a commands receiving member (240) in the apparatus (100) configured to receive commands based on the qualified data at a given point of time from the external unit (135) by means of low power signals, and the controller (125) being configured to integrate the commands; and
a mobile device (230) operable to read the unique identification means (140).

Finally, in a third aspect of the invention, there is provided a method of reducing or controlling water consumption in bathrooms, the method comprising the steps of:
providing an apparatus (100) being configured to receive data from one or more water consumption locations in a bathroom, the apparatus comprising a housing (105) including one or more sensors (107), a controller (125), a transmitting member (130), and a unique identification means (140);
detecting a movement in a certain distance from the apparatus (100) in a bathroom as a first indication of activity in the bathroom by means of a motion sensor (110), detecting a sound of running water from one or more water consumption locations in a bathroom as a second indication of activity in the bathroom by means of an audio sensor (115), optionally detecting the ambient humidity in a bathroom as a third indication of activity in the bathroom by means of a humidity sensor (120), and optionally detecting ambient temperature by means of an ambient temperature sensor (200);
qualifying at least a part of the data received from the one or more sensors (107) by means of an algorithm to obtain qualified data;
transmitting the qualified data at a given point of time to an external unit (135), optionally via a mobile device (230), by means of low power signals; and
subjecting output data based on the qualified data for reducing or controlling water consumption in the bathroom to a unique identification means (140) being readable or scannable by a mobile device (230).

One of the advantages of the present invention is that water consumption may be reduced for water installations in bathroom, such as showers, by minutes or even seconds. Reducing water consumption in showers by minutes or even seconds would generally accomplish a milestone in energy consumption balance budgets.

Generally, use of sensors for detection of human presence and interaction in bathrooms is difficult as it requires recognition of multiple variables in order to identify and qualify such presence and interaction, particularly if an intelligent solution is to be established for behavioral interaction.

According to the invention, there is provided one or more sensors (107) including a motion sensor (110) configured to detect a movement in a certain distance from the apparatus in a bathroom as a first indication of activity in the bathroom, and an audio sensor (115) configured to monitor a sound of running water from one or more water consumption locations in a bathroom as a second indication of activity in the bathroom.

In the present context “as a first indication of activity in the bathroom” is intended to mean a first sequence of identification means, and “as a second indication of activity in the bathroom” is intended to mean a second sequence of identification means. Hence, the apparatus of the invention detects motion as the first activity and audio as the next activity. However, as time continues both sensors may be in action with an overlap and may end detecting at different points of time. These precise calculations of activity are incorporated in an algorithm and/or preset in the controller.

As a third step, a humidity sensor configured to monitor the ambient humidity in a bathroom may optionally be applied as a third indication of activity in the bathroom. In some embodiments, this third option is preferred, such as in the case where data from the first two sensors is to be validated as properly reflecting water consumption at a location in the bathroom. Also, this third sensor may be used to detect climate conditions in a bathroom, preferably in connection with an optional ambient temperature sensor. This may prevent formation of mold in the bathroom or other derived problems to be monitored.

Also, for the optional third humidity sensor, the motion sensor and audio sensor may continue detecting, and preferably do so. In the present context “as a third indication of activity in the bathroom” is intended to mean a third sequence of identification means. Hence, the apparatus of the invention optionally detects humidity as the third activity, while still detecting motion and/or audio activity. However, as time continues all sensors may be in action with an overlap and may end detecting at different points of time. These precise calculations of activity is incorporated in an algorithm or preset in the controller.

According to the invention, the housing of the apparatus comprises a controller (125) configured to qualify at least a part of the data received from the one or more sensors (107) by means of an algorithm to obtain qualified data. This controller may be a CPU processor or other means available to perform qualification of data by means of an algorithm. The housing may also include storage means, such as a memory card that allows data collection for a longer period of time before further transmission. Further submission may be activated when needed or at predetermined time intervals, such as one time a day, two times a day, each two hours, or the like. It may also be real time transmission. The controller receives signals from the sensoring means and may do so directly, or through other components in the apparatus.

The term “qualify” is intended to mean quality controlling of real data for human interaction by means of an algorithm, such as iteration or numerical analysis of data received. An algorithm may not be needed for all the sensors involved in data collection, but may be used for only one sensoring means, such as the audio sensor, two or more sensoring means. Noise from other sources may intervene with noise from for instance a shower and may preferably be discarded. In this instance, the algorithm is configured to fractionate the sound of running water from background noise and even sources with very similar frequency as running water, such as a hair dryer. The controller may provide a first set of qualified data which later may be supplemented with further qualification, for instance in an external unit. Accordingly, the term “qualified” in the present context is intended to mean motion data and audio data that involves human interaction, optionally humidity data that involves human interaction.

Generally, the apparatus and system is set up to estimate water consumption as a function of time, e.g., the total time of each shower or incidences of running water of a certain time length. One option is to have the display in the apparatus visualizing the water consumption time for a given water installation, such as a shower. In this case, real time data may be available for inspection directly in addition to data transmitted to an external unit. These data may be qualified by the controller before they are visualized on the display. In the alternative, these data visualized on the display may be raw data that has not undergone any qualification process.

According to the invention, a transmitting member (130), such as an antenna, configured to transmit the qualified data at a given point of time to an external unit (135) is provided in the housing of the apparatus which uses low power signals. Optionally the qualified data may also be processed via a mobile device (230) by means of low power signals to an external unit. In the present context the “external unit” may be a computing device or simply an accessible cloud storage solution, optionally further comprising a dashboard for tracking and monitoring one or more bathrooms, such as bathrooms in a building, hotel or building complex. Further qualification of data may be provided by the external unit, such as confirmation of audio and motion by humidity data which may be somehow delayed due to a relatively slow increase in humidity upon water installation usage. This option may serve to be a further quality check of data. Further qualification may preferably be made externally but may also be conducted in the controller in a pre-setup.

In the present context a “bathroom” should be understood in a broad sense. While showers may usually be located in rooms dedicated for the purpose, water taps or toilets may be located in rooms that are not particularly dedicated for the purpose. These rooms include hotel rooms with a wash and a water tap located in a main room or other rooms that are not addressed for the purpose. Also, bathrooms may be understood as rooms dedicated for toilets and water taps, and for rooms only dedicated for showers. Additionally, shower rooms in public places such as swimming facilities fall under the definition of “bathroom” in the present context. A person skilled in the art would provide a reasonable interpretation in view of the present invention.

In the present context, when “low power signals” is mentioned, this may include narrow band low power signals or other low power signals, such as Bluetooth. In the present context, Wi-Fi is not intended for the present invention as the battery requirements are too high. Use of low power signals were seen by the inventors to be a huge advantage according to the invention, which may allow the apparatus to be placed in locations without constant wired energy supply, even in remote places hardly accessible for maintenance.

Communication from the apparatus to the external unit is preferably made with narrow band low power signals. Communication via a mobile device to the external apparatus is preferably made with Bluetooth but does not necessarily need to be so but can also be with other low power signals. The use of low power signals in the present invention was not expected by the inventors to give this much added benefit that was seen in the resulting product. Accordingly, energy consuming Wi-Fi may be avoided and frequent charging of batteries may be avoided. Also, the system may be particularly vulnerable by using more energy consuming sources due to the relative humid conditions in bathrooms, and since the apparatus may be susceptible to direct water exposure from water installations.

In some embodiments of the invention, the unique identification means (140) is a matrix barcode, such as a quick response (QR) code, or a near-field communication (NFC) chip. Other means are also within the scope of the invention. In some embodiments, a quick response (QR) code is preferred. The matrix barcode may be visible on the housing but may in a preferred embodiment be readable or scannable by a mobile device and located on a display under a transparent portion of the cover of the housing.

In the present context, when the apparatus is mentioned to comprise a unique identification means, such as a QR code, it may we visible and readable at any given point and time but may also be visible and readable for only a period of time. For instance, the display of the present invention may show the clock and/or water consumption time, and when needed also the QR code. An NFC chip, if present, may be scannable at any given point of time and may be hidden below the cover.

The great advantage of using identification means in the present invention was a surprise to the inventors. It was not expected that for instance a QR code was even possible to apply in the apparatus and would be possible to apply without compromising the function of the apparatus. Also, the QR code provides for a very flexible solution and may be scannable or readable on the apparatus directly by a user of the water installation in the bathroom, without using an app. The output data based on qualified data may be directly presented to the user and applied directly in the bathroom.

The unique code makes it possible to apply a central unit, the external unit, that may collect qualified data from a number of devices and allocate the specific qualified data for the specific bathroom when needed. Hence, the system according to the invention and the apparatus as such, provide a huge benefit compared to prior art systems, where user interaction directly in a bathroom is usually not provided for.

In some embodiments of the invention, the controller (125) is configured to qualify at least a part of the data received from the motion sensor (110) as qualified motion data by means of an algorithm based on a reference motion standard. In this embodiment, the motion sensor may qualify data by distance to a moving object, or it may use artificial intelligence to detect a moving object.

In some embodiments of the invention, the controller (125) is configured to qualify at least a part of the data received from the audio sensor (115) as qualified audio data by means of an algorithm based on a reference audio standard. In this embodiment, noise from the surroundings may be filtered in order to detect running water. Since the noise of running water from various showers may be different, qualification may also take this aspect into account.

Also, noise that has a similar characteristics to running water may be filtered and qualification may take this into account as well. For instance, noise from a hair dryer was seen by the inventors to cause huge problems in differentiation from the noise of running water. Qualification of data in this respect is also accounted for as well as other external noise sources. It was a surprise to the inventors that it was possible in the end to use an audio sensor to detect running water due to the difficulties in filtering noise from other sources.

In some embodiments of the invention, the controller (125) is configured to both qualify at least a part of the data received from the motion sensor (110) as qualified motion data by means of an algorithm based on a reference motion standard and is configured to qualify at least a part of the data received from the audio sensor (115) as qualified audio data by means of an algorithm based on a reference audio standard

In some embodiments of the invention, the transmitting member (130) is configured to transmit motion data and audio data at a given point of time if the motion data and the audio data are qualified by the controller (125). In this embodiment, data is not transmitted if both motion data and audio data is disqualified, which may be the case when human interaction is calculated not to be present.

In some embodiments of the invention, the transmitting member (130) is configured to transmit humidity data at a given point of time if the humidity data is qualified by the controller (125).

In some embodiments of the invention, the transmitting member (130) is configured to transmit humidity data at a given point of time if the humidity data is qualified by the controller (125). In this embodiment, humidity data is not transmitted if both are disqualified, which may be the case when human interaction is calculated not to be present.

In some embodiments of the invention, the identification means (140) is configured to include output data if motion data and audio data at a given point of time are qualified by the controller (125), and humidity data is qualified by the controller (125) or the external unit (135) to encompass a predetermined change in humidity as the third indication of activity in the bathroom.

In some embodiments of the invention, the transmitting member (130) is configured to transmit motion data, audio data and humidity data at a given point of time if the motion data, the audio data, and the humidity data are qualified by the controller (125).

In some embodiments of the invention, the one or more sensors (107) includes an ambient temperature sensor (200).

In some embodiments of the invention, the transmitting member (130) is configured to transmit temperature data at a given point of time if the temperature data is qualified by the controller (125).

In some embodiments of the invention, the identification means (140) is configured to include output data if motion data and audio data at a given point of time are qualified by the controller (125), humidity data is qualified by the controller (125) or the external unit (135) to encompass a predetermined change in humidity as the third indication of activity in the bathroom, and temperature data is qualified by the controller (125) or the external unit (135).

In some embodiments of the invention, the transmitting member (130) is configured to transmit motion data, audio data, humidity data and temperature data at a given point of time if the motion data, the audio data, the humidity data, and the temperature data are qualified by the controller (125).

In some embodiments of the invention, the motion sensor (110) is a passive infrared sensor (PIR sensor) located below the level of the surface of the housing. Since privacy is of outmost importance in bathroom, the inventors of the present invention developed means for hiding the motion sensor. The solution in this embodiment is highly beneficial and unexpectedly provides for accurate motion detection, even though the sensor is not as exposed as in prior art solutions.

In some embodiments of the invention, data are integrated by the controller (125) or the external unit (135).

In some embodiments of the invention, motion data, audio data, and humidity data are integrated by the controller (125) or the external unit (135).

In some embodiments of the invention, motion data, audio data, humidity data and temperature data are integrated by the controller (125) or the external unit (135).

In some embodiments of the invention, the one or more sensors (107) includes a carbon dioxide sensor.

In some embodiments of the invention, the transmitting member (130) is configured to transmit carbon dioxide data at a given point of time if the carbon dioxide data is qualified by the controller (125).

In some embodiments of the invention, the identification means (140) is configured to include output data if carbon dioxide data at a given point of time is qualified by the controller (125) or the external unit (135).

In some embodiments of the invention, the transmitting member (130) is configured to transmit motion data, audio data, humidity data, temperature data and carbon dioxide data at a given point of time if the motion data, the audio data, the humidity data, the temperature data, and the carbon dioxide data are qualified by the controller (125).

In some embodiments of the invention, the one or more water consumption locations in a bathroom comprises the space defined by the inside of the walls of the bathroom, excluding locations within or outside the walls of the bathroom.

In some embodiments of the invention, the housing (105) further comprises a commands receiving member (145) configured to receive commands based on the qualified data at a given point of time from the external unit (135) by means of low power signals, and the controller (125) is configured to integrate the commands. In this embodiment, the controller may be upgraded to perform better based on qualification of data, preferably based on a huge set of data, such as metadata. In the present context “metadata” is intended to mean integration of data for more than one bathroom, such as for numerous bathrooms. The commands in the present context may serve to change the preset conditions in the controller or may even provide learning algorithms to the controller for better performing qualification of data.

The external unit (135) may in addition to the controller in the apparatus comprise an algorithm that may serve to improve the general function of the apparatus. For instance, the algorithm may work to improve the qualified data already received from the controller and send it back to the controller for improved performance of the apparatus. This may be by means of a numeral analysis but also artificial intelligence. Typically, such commands are received by a commands receiving member (145) in the apparatus. One example of commands may be commands for improving the apparatus' water consumption performance or ability to detect water consumption over time.

In some embodiments of the invention, the apparatus comprises a bracket (150) with a magnetic member (155), and the housing (105) comprises a hall sensor (160) to detect the magnetic member (155) upon placing the housing in the bracket (150) whereby the apparatus (100) is activated. In this embodiment, installation is made very easy for a user. Essentially, when the apparatus is mounted on the bracket, the system automatically starts up. Since the apparatus may preferably have a build-in chip for external communication, the system may automatically go online and be communicably connected with the external unit.

In some embodiments of the invention, the housing of the apparatus comprises a front cover (170) including a transparent centrally arranged portion (175) facing the front of said cover (170) that allows visibility to a display (180) positioned below the transparent centrally arranged portion (175), and a portion with a substantially thinner wall thickness (185) that allows functioning of the motion sensor (110) located below the level of the surface of the housing.

In some embodiments of the invention, the housing of the apparatus comprises a back cover (190) connectable and sealable with the front cover (170), the back cover (190) encasing a printing board (195), the printing board (195) including the display (180) operable to visualize the unique matrix barcode (140), the one and more sensors, the controller (125), and the transmitting member (130).

In some embodiments of the invention, the housing of the apparatus comprises a back plate (210) connectable and sealable with the back cover (190), the back plate and the back cover encasing a power supply member (220) for operating the apparatus.

In some embodiments of the invention, the output data scannable or readable on the apparatus includes one or more of water-saving tips, shower statistics, water tap statistics, toilet usage statistics, running water statistics and running water alerts. The output data based on the qualified data may include further statistics. Additionally, the output data may also serve to nudge a user to use water installations differently.

With respect to the second aspect of the invention, the system may comprise an external unit (135) configured to receive qualified data from the apparatus (100) at a given point of time, optionally via a mobile device (230), by means of low power signals, and being configured to process data based on the qualified data and/or based on further qualification of data, such as humidity data, and configured to incorporate output data in a unique identification means (140) being readable by a mobile device (230) on the apparatus (100) for reducing or controlling water consumption in a bathroom.

Optionally, the system may comprise a commands receiving member (240) in the apparatus (100) configured to receive commands based on the qualified data at a given point of time from the external unit (135) by means of low power signals, and the controller (125) being configured to integrate the commands.

Additionally, the system may comprise a mobile device (230) operable to read the unique identification means (140). In the present context a “mobile device” may be any handheld means for detecting the unique identification means, such as a mobile phone or a tablet.

In some embodiments of the invention, the system further includes one or more external sensors (235) communicably coupled to the apparatus (100), optionally by Bluetooth, and further including a sensor receiving member (245) in the apparatus (100) configured to receive data from the one or more external sensors (235), and the controller (125) being configured to integrate the data. The data may be further transmitted to an external unit and/or a mobile device, as is the case for the qualified data.

In some embodiments of the invention, the one or more external sensors (235) include one or more external temperature sensors mounted to a water inlet (240) of a local installation (250) in the bathroom, including one or more inlets of a toilet (255), a water tap (257) or a shower (260), and the one or more external temperature sensors being configured to detect a temperature difference of the water in the inlet (240) compared to ambient temperature over time, whereby running water may be detected. This may be done by ultrasonic means or specific temperature sensors mounted on or around water vessels close to the water installation in the bathroom.

In some embodiments of the invention, the one or more external sensors (235) include an external temperature sensor mounted to a water inlet of a first local installation in the bathroom, such as a toilet (255), in combination with an external audio sensor in proximity to a water inlet of a second local installation in the bathroom and/or an external temperature sensor mounted to the water inlet of a second local installation in the bathroom, such as a water tap (257), whereby coordinated usage of both installations may be detected.

In some embodiments of the invention, the system further includes an alert for running water to the mobile device (230) and/or to the external unit (135). In this embodiment, running water in a toilet may be detected early and transmitted to a mobile device and/or an external unit.

In some embodiments of the invention, the apparatus (100) is included in a plurality of bathrooms, and the external unit (135) is configured to process metadata based on qualified data from a plurality of bathrooms, optionally including said metadata in the unique identification means (140) and/or a central dashboard (142).

With respect to the second aspect of the invention, the method may comprise a step of detecting a movement in a certain distance from the apparatus (100) in a bathroom as a first indication of activity in the bathroom by means of a motion sensor (110), detecting a sound of running water from one or more water consumption locations in a bathroom as a second indication of activity in the bathroom by means of an audio sensor (115), optionally detecting the ambient humidity in a bathroom as a third indication of activity in the bathroom by means of a humidity sensor (120), and optionally detecting ambient temperature by means of an ambient temperature sensor (200).

In some embodiments of the invention, the one or more external sensors (235) is located within the space defined by the inside of the walls of the bathroom, excluding locations within or outside the walls of the bathroom.

Additionally, the method may comprise a step of qualifying at least a part of the data received from the one or more sensors (107) by means of an algorithm to obtain qualified data. Further, the method may comprise a step of transmitting the qualified data at a given point of time to an external unit (135), optionally via a mobile device (230), by means of low power signals. Finally, the method may comprise a step of subjecting output data based on the qualified data for reducing or controlling water consumption in the bathroom to a unique identification means (140) being readable or scannable by a mobile device (230).

It is to be understood that elements of the system and method according to the invention may be applied in combination with the apparatus according to the invention. Hence, specific elements of the method may be applied and combined with other elements of the apparatus as described herein.

In some embodiments of the invention, the audio sensor (115) is protected from water droplets in the apparatus (100) by means of a trap (270) that serves to avoid water interference without substantially reducing audio signals, the trap (270) comprising a U-shaped air channel (275) starting from the face of a bottom portion (276) of the apparatus (100) and extending towards the audio sensor (115). This trap was surprisingly seen to work and provide added benefits in the harsh humid environment of bathrooms. For example, water droplets exposed to the apparatus was seen to be trapped in the U-shaped channel of the apparatus. This embodiment makes it possible to avoid filters that are usually seen as reducing sound quality and the sound picture in general.

In some embodiments of the invention, the humidity sensor (120) is protected from water droplets in the apparatus (100) by means of a trap (270) that serves to avoid water interference without substantially reducing audio signals, the trap (270) comprising a U-shaped air channel (285) starting from the face of a bottom portion (276) of the apparatus (100) and extending towards the humidity sensor (120). This trap was surprisingly seen to work and provide added benefits in the harsh humid environment of bathrooms. For example, water droplets exposed to the apparatus was seen to be trapped in the U-shaped channel of the apparatus. This embodiment makes it possible to avoid interference with the humidity sensor.

In some embodiments of the invention, a bracket (150) of the apparatus (100) is fixed to the housing (105) by means of a connecting member (290), such as a screw, adjusted to secure that the audio sensor (115) in the housing (105) is tuned for optimized sound picture. This embodiment was seen as a very huge advantage in that the audio sensor according to the invention may need a precise positioning in order to deliver the most advantageous sound picture. The connecting member may both be used to attach the bracket to the apparatus and at the same time be used to adjust positioning of the apparatus in a correct position.

The invention will be understood in greater detail with reference to the following figures that serve to illustrate certain particular embodiments of the invention by way of example.

### BRIEF DESCRIPTION OF DRAWINGS

FIG. 1 is a perspective view of the apparatus according to the invention, illustrating the various visible parts on top of the apparatus in an unfolded position.

FIG. 2 is top view of the apparatus with the front cover unmounted, and a printing board with a display centrally arranged and positioned in the back cover.

FIG. 3 illustrates the back of the apparatus in the top section, where the back cover and the back plate mounted to the back cover, as well as various elements on the back side of the apparatus. The bottom section of the drawing illustrates the back of the apparatus, where the back cover is made transparent in order to see the printing board underneath.

FIG. 4 is a perspective view of the bracket to be mounted on a wall and the apparatus according to the invention.

FIG. 5 is a cross-sectional view of the apparatus, where the U-shaped trap is illustrated for the audio sensor in the apparatus.

FIG. 6 is a cross-sectional view of the apparatus, where the U-shaped trap is illustrated for the humidity sensor in the apparatus.

FIG. 7 is an expanded view of the apparatus, including the front cover, printing board and back cover.

FIG. 8 is an expanded view of the apparatus, including the front cover mounted with the back cover, the energy storage means (battery), back plate and the bracket.

FIG. 9 is an overview of the system according to the invention, including the apparatus according to the invention. The large, squared box illustrates the apparatus.

### DETAILED DESCRIPTION

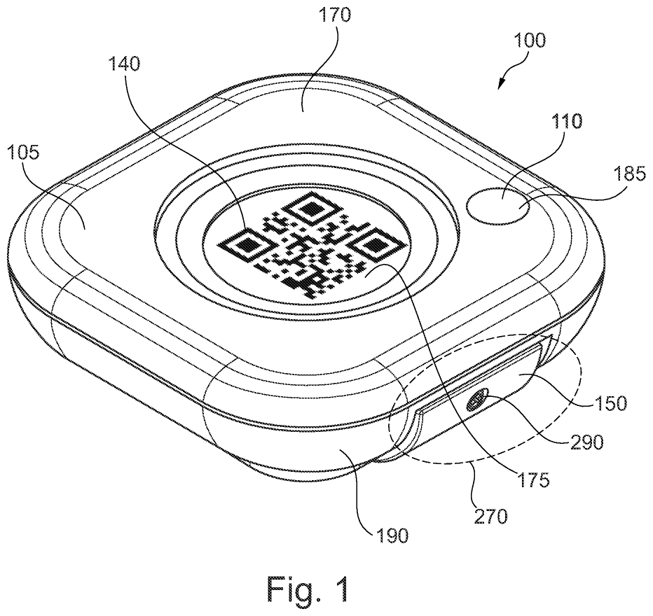

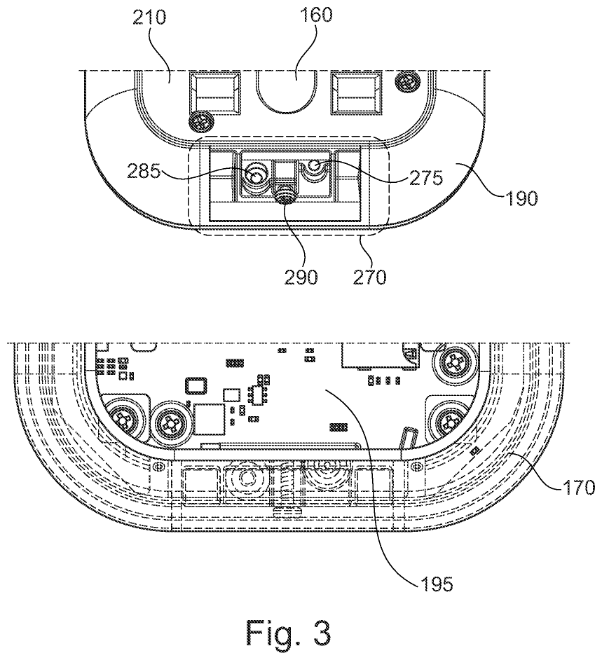

Referring now to FIGS. 1, 2, 3, 5, 6, 7 and 8 of the drawings, there is provided an apparatus (100) for reducing or controlling water consumption in bathrooms, the apparatus (100) comprising a housing (105) and being configured to receive data from one or more water consumption locations in a bathroom. The housing (105) comprises one or more sensors (107) including a motion sensor (110) configured to detect a movement in a certain distance from the apparatus in a bathroom as a first indication of activity in the bathroom, an audio sensor (115) configured to monitor a sound of running water from one or more water consumption locations in a bathroom as a second indication of activity in the bathroom, optionally a humidity sensor (120) configured to monitor the ambient humidity in a bathroom as a third indication of activity in the bathroom, and optionally an ambient temperature sensor (200).

Further, the apparatus comprises a controller (125) configured to qualify at least a part of the data received from the one or more sensors (107) by means of an algorithm to obtain qualified data. Also, the apparatus comprises a transmitting member (130) configured to transmit the qualified data at a given point of time to an external unit (135), optionally via a mobile device (230), by means of low power signals. Finally, as illustrated the apparatus comprises a unique identification means (140) being readable or scannable by a mobile device and configured to include output data based on the qualified data for reducing or controlling water consumption in the bathroom.

Referring now to FIG. 1 of the drawings there is provided a unique identification means (140) is a matrix barcode, such as a quick response (QR) code, or a near-field communication (NFC) chip.

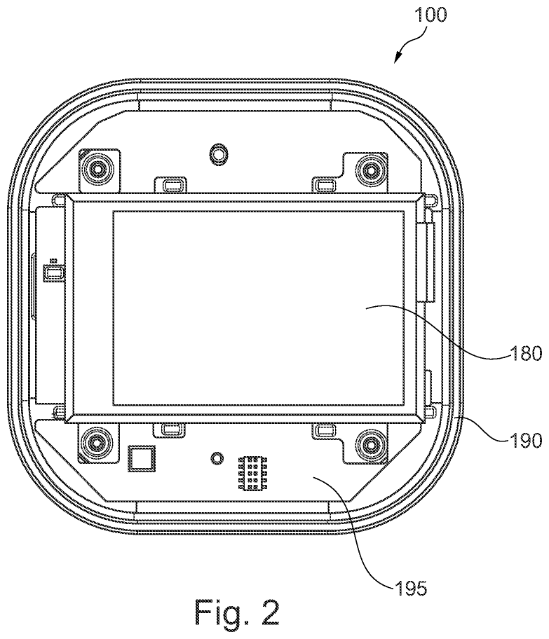

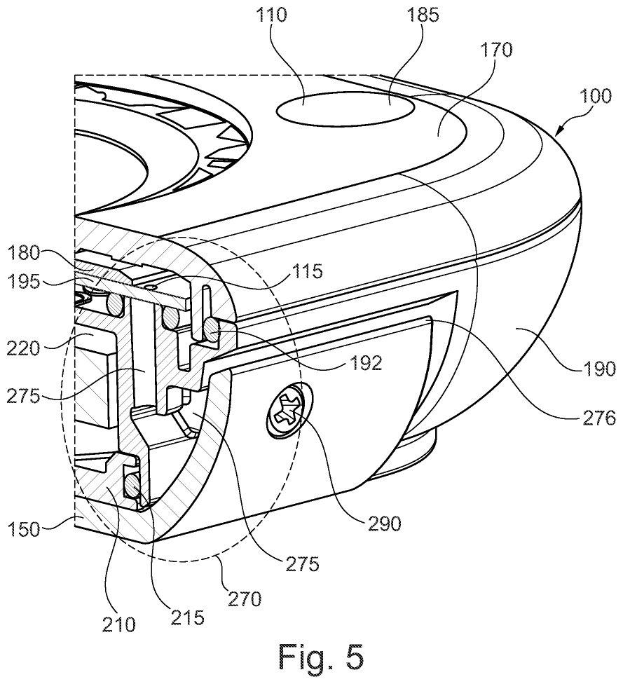

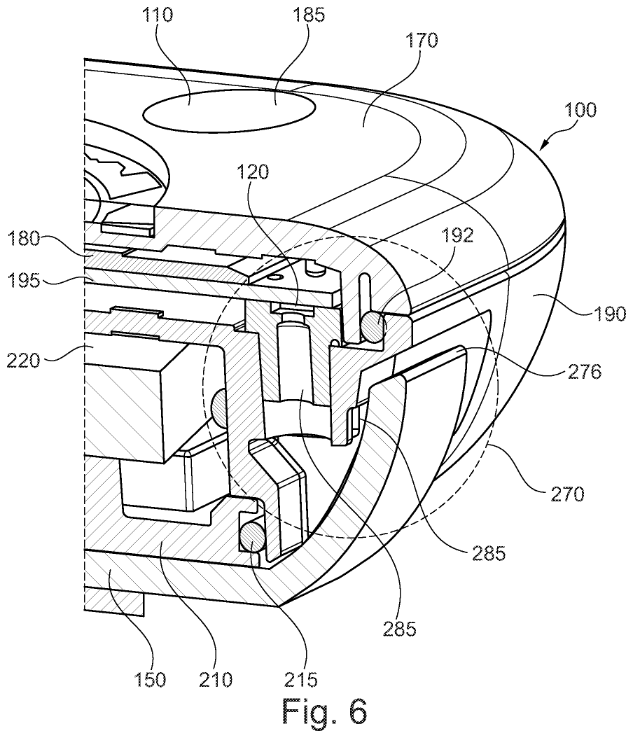

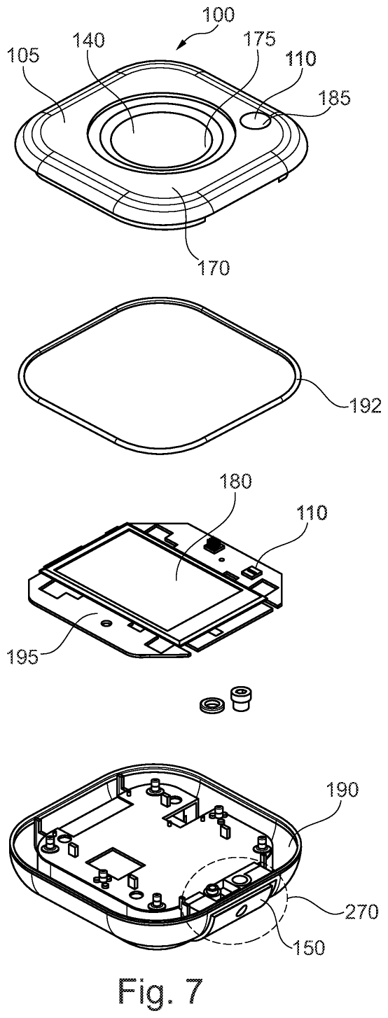

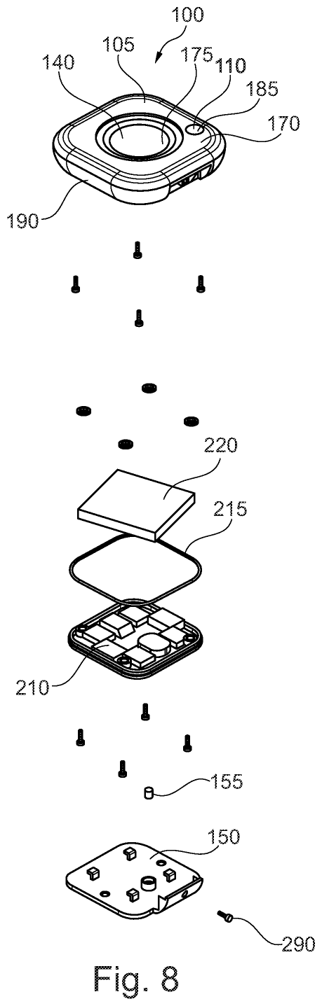

The apparatus illustrated comprises a controller (125) configured to qualify at least a part of the data received from the motion sensor (110) as qualified motion data by means of an algorithm based on a reference motion standard. The controller is located on a printing board of the apparatus, which printing board is illustrated in FIG. 2, 3, partly in FIGS. 5 and 6, in FIGS. 7 and 8.

The controller (125) is configured to qualify at least a part of the data received from the audio sensor (115) as qualified audio data by means of an algorithm based on a reference audio standard. Further, the controller (125) is configured to qualify at least a part of the data received from the audio sensor (115) as qualified audio data by means of an algorithm based on a reference audio standard.

The apparatus (100) illustrated comprises a transmitting member (130) configured to transmit motion data and audio data at a given point of time if the motion data and the audio data are qualified by the controller (125). The apparatus (100) illustrated is also configured to transmit humidity data at a given point of time if the humidity data is qualified by the controller (125).

With respect to the identification means (140) as illustrated in FIG. 1, the identification means (140) is configured to include output data if motion data and audio data at a given point of time are qualified by the controller (125), and humidity data is qualified by the controller (125) or the external unit (135) to encompass a predetermined change in humidity as the third indication of activity in the bathroom.

FIGS. 1, 5, 6, 7 and 8 illustrates the motion sensor (110) as a passive infrared sensor (PIR sensor) located below the level of the surface of the housing. The motion sensor is a PIR as here illustrated but may also be another type of motion sensor according to the invention.

The apparatus (100) illustrated may further comprise (not illustrated here), a commands receiving member (145) configured to receive commands based on the qualified data at a given point of time from the external unit (135) by means of low power signals, and the controller (125) is configured to integrate the commands.

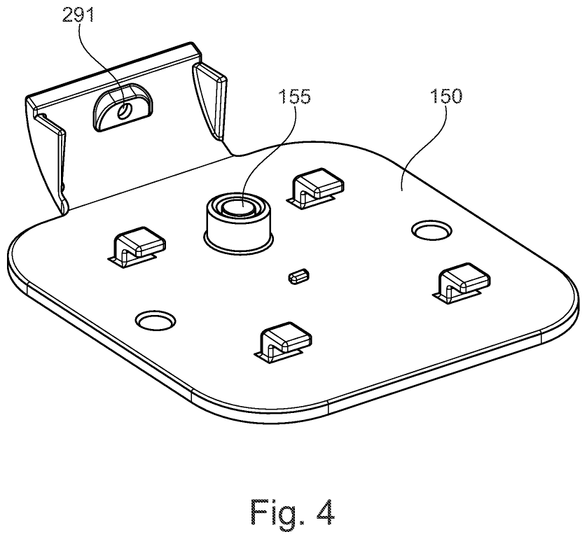

Referring now to FIG. 4, partly FIGS. 5, 6 and 7, and FIG. 8 of the drawings, there is provided a bracket (150) with a magnetic member (155), and the housing (105) comprises a hall sensor (160) to detect the magnetic member (155) upon placing the housing in the bracket (150) whereby the apparatus (100) is activated.

Referring further to FIGS. 1, 2, 3, 5, 6, 7 and 8 of the drawings, there is provided illustrations of the housing of the apparatus, comprising a front cover (170) including a transparent centrally arranged portion (175) facing the front of said cover (170) that allows visibility to a display (180) positioned below the transparent centrally arranged portion (175), and a portion with a substantially thinner wall thickness (185) that allows functioning of the motion sensor (110) located below the level of the surface of the housing;
a back cover (190) connectable and sealable with the front cover (170), the back cover (190) encasing a printing board (195), the printing board (195) including the display (180) operable to visualize the unique matrix barcode (140), the one and more sensors, the controller (125), and the transmitting member (130);
a back plate (210) connectable and sealable with the back cover (190), the back plate and the back cover encasing a power supply member (220) for operating the apparatus.

As illustrated in FIG. 5, and indicated in FIGS. 1, 3 and 7, the audio sensor (115) is protected from water droplets in the apparatus (100) by means of a trap (270) that serves to avoid water interference without substantially reducing audio signals, the trap (270) comprising a U-shaped air channel (275) starting from the face of a bottom portion (276) of the apparatus (100) and extending towards the audio sensor (115).

As illustrated in FIG. 6, and indicated in FIGS. 1, 3 and 7, the humidity sensor (120) is protected from water droplets in the apparatus (100) by means of a trap (270) that serves to avoid water interference without substantially reducing audio signals, the trap (270) comprising a U-shaped air channel (285) starting from the face of a bottom portion (276) of the apparatus (100) and extending towards the humidity sensor (120).

Furthermore, as illustrated in FIG. 1, 3, and indicated in FIG. 4 with a hole (291), 5 and 8, there is provided a bracket (150) of the apparatus (100) fixed to the housing (105) by means of a connecting member (290), such as a screw, adjusted to secure that the audio sensor (115) in the housing (105) is tuned for optimized sound picture.

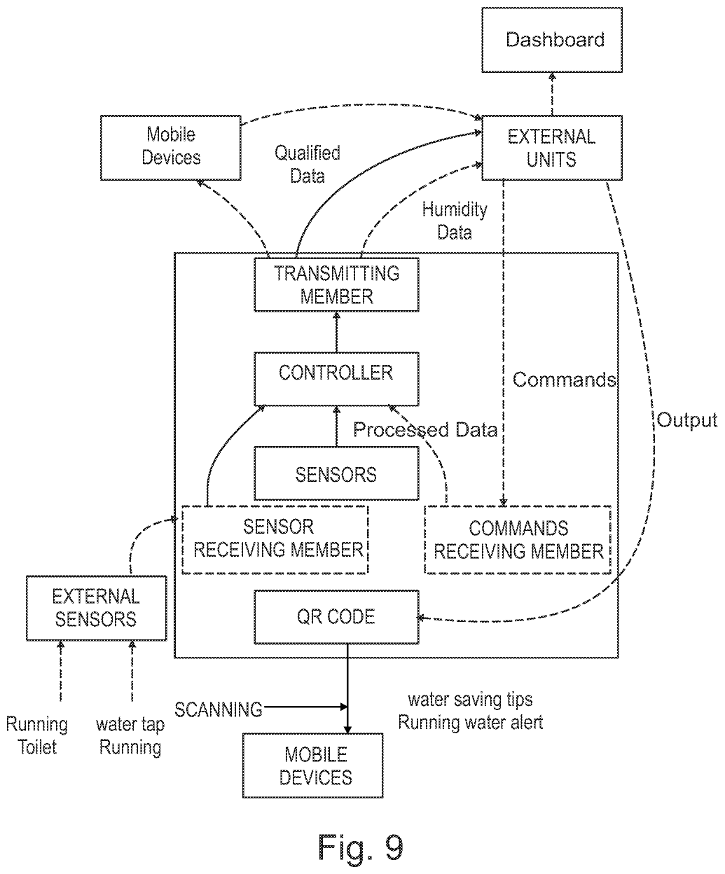

Turning to the system according to the invention, this is illustrated in FIG. 9. Here, it can also be seen how the various elements work together in the system. The system is illustrated with one or more external sensors (235) communicably coupled to the apparatus (100), optionally by Bluetooth, and further including a sensor receiving member (245) in the apparatus (100) configured to receive data from the one or more external sensors (235), and the controller (125) being configured to integrate the data.

The system may include one or more external sensors (235) include one or more external temperature sensors mounted to a water inlet (240) of a local installation (250) in the bathroom, including one or more inlets of a toilet (255), a water tap (257) or a shower (260), and the one or more external temperature sensors being configured to detect a temperature difference of the water in the inlet (240) compared to ambient temperature over time, whereby running water may be detected. Further, the system may include one or more external sensors (235) include an external temperature sensor mounted to a water inlet of a first local installation in the bathroom, such as a toilet (255), in combination with an external audio sensor in proximity to a water inlet of a second local installation in the bathroom and/or an external temperature sensor mounted to the water inlet of a second local installation in the bathroom, such as a water tap (257), whereby coordinated usage of both installations may be detected.

Further, the system may include an alert for running water to the mobile device (230) and/or to the external unit (135), such as one or more alerts (not illustrated).

The apparatus of the system may especially be adapted to be included in a plurality of bathrooms (not illustrated), and the external unit (135) is configured to process metadata based on qualified data from a plurality of bathrooms, optionally including said metadata in the unique identification means (140) and/or a central dashboard (142).

Turning to FIG. 9, the system according to the invention is illustrated and also provides an overview of the method according to the invention. The large, squared box illustrates the apparatus and the elements described in detail in the previous sections. The sensors in the apparatus include an audio sensor and a motion sensor, and optionally a humidity sensor and/or a temperature sensor. Data from the sensors in the apparatus are delivered to the controller where qualification of the data is performed. Data may further be stored in storing means in the apparatus until they are transmitted from the apparatus to an external unit, optionally via a mobile device by low power means, such as Bluetooth. In the presently preferred set-up, qualified data from the controller, optionally via other elements in the apparatus, is transmitted to an external unit by use of narrow band low power signals. Humidity data may also be transmitted to the external unit and be transmitted independently from motion and audio data.

Once, the qualified data is received by the external unit, further qualification may be performed by the external unit in order to achieve output data with high quality. In some embodiments of the invention, data from a number of bathrooms may be transmitted to the external unit and further conveyed to a dashboard where full control of various bathrooms may be performed. The high-quality output is then optionally further categorised or optimized.

If necessary, or as part of optimization procedures, commands may be transmitted back to a commands receiving member in the apparatus in order to optimize the system or pre-set requirements in the controller. For instance, more intelligent algorithms may be provided based on complete data from various bathrooms. Metadata may then be used for optimization of the systems. It may also be small adjustments or reference data that may be transmitted to the controller and incorporated in the apparatus.

Finally, output data may be directly scanned by a mobile device on the apparatus and may provide output data includes one or more of water-saving tips, shower statistics, water tap statistics, toilet usage statistics, running water statistics and running water alerts.

As a separate option that may be integrated in the system is external sensors allocated to specific water installations in the bathrooms. These external sensors may be directly mounted to water pipes in close proximity to the water installations and may provide valuable data to the apparatus. Preferably, these sensors use Bluetooth but other low power means may also be used. Specifically, one option is to apply these external sensors to a toilet in order to detect if the toilet is running with water. Also, an external sensor may be provided to or close to a water tap, such as a audio sensor, that may detect running water from the water tap. In combination, if both external sensors are provided, it may detect valuable information, such as identifying derived symptoms among user of the bathroom, including diseases such as incontinence and cystitis.

1. An apparatus for reducing or controlling water consumption in bathrooms, the apparatus comprising a housing and being configured to receive data from one or more water consumption locations in a bathroom, the housing comprising:
one or more sensors including a motion sensor configured to detect a movement in a threshold distance from the apparatus in a bathroom as a first indication of activity in the bathroom, and an audio sensor configured to monitor a sound of running water from one or more water consumption locations in a bathroom as a second indication of activity in the bathroom, optionally a humidity sensor configured to monitor the ambient humidity in a bathroom as a third indication of activity in the bathroom, and optionally an ambient temperature sensor;
a controller configured to qualify at least a part of the data received from the one or more sensors by an algorithm to obtain qualified data;
a transmitting member configured to transmit the qualified data at a given point of time to an external unit, optionally via a mobile device, by low power signals; and
a unique identification mechanism being readable or scannable by a mobile device and configured to include output data based on the qualified data for reducing or controlling water consumption in the bathroom.

2. The apparatus according to claim 1, wherein the unique identification mechanism is a matrix barcode.

3. The apparatus according to claim 1, wherein the controller is configured to qualify at least a part of the data received from the motion sensor as qualified motion data by an algorithm based on a reference motion standard.

4. The apparatus according to claim 1, wherein the controller is configured to qualify at least a part of the data received from the audio sensor as qualified audio data by an algorithm based on a reference audio standard.

5. The apparatus according to claim 1, wherein the controller is configured to both qualify at least a part of the data received from the motion sensor as qualified motion data by an algorithm based on a reference motion standard and is configured to qualify at least a part of the data received from the audio sensor as qualified audio data by an algorithm based on a reference audio standard.

6. The apparatus according to claim 1, wherein the transmitting member is configured to transmit motion data and audio data at a given point of time if the motion data and the audio data are qualified by the controller.

7. The apparatus according to claim 1, wherein the one or more sensors includes a humidity sensor configured to monitor the ambient humidity in a bathroom as a third indication of activity in the bathroom.

8. The apparatus according to claim 7, wherein the transmitting member is configured to transmit humidity data at a given point of time if the humidity data is qualified by the controller.

9. The apparatus according to claim 7, wherein the identification mechanism is configured to include output data if motion data and audio data at a given point of time are qualified by the controller, and humidity data is qualified by the controller or the external unit to encompass a predetermined change in humidity as the third indication of activity in the bathroom.

10. The apparatus according to claim 8, wherein the transmitting member is configured to transmit motion data, audio data and humidity data at a given point of time if the motion data, the audio data, and the humidity data are qualified by the controller.

11. The apparatus according to claim 1, wherein the one or more sensors includes an ambient temperature sensor.

12. The apparatus according to claim 11, wherein the transmitting member is configured to transmit temperature data at a given point of time if the temperature data is qualified by the controller.

13. The apparatus according to claim 11, wherein the identification mechanism is configured to include output data if motion data and audio data at a given point of time are qualified by the controller, humidity data is qualified by the controller or the external unit to encompass a predetermined change in humidity as the third indication of activity in the bathroom, and temperature data is qualified by the controller or the external unit.

14. The apparatus according to claim 12, wherein the transmitting member is configured to transmit motion data, audio data, humidity data and temperature data at a given point of time if the motion data, the audio data, the humidity data, and the temperature data are qualified by the controller.

15. The apparatus according to claim 1, wherein the motion sensor is a passive infrared sensor (PIR sensor) located below the level of the surface of the housing.

16. The apparatus according to claim 1, wherein data are integrated by the controller or the external unit.

17. The apparatus according to claim 1, wherein motion data, audio data, and humidity data are integrated by the controller or the external unit.

18. The apparatus according to claim 1, wherein motion data, audio data, humidity data and temperature data are integrated by the controller or the external unit.

19. The apparatus according to claim 1, wherein the one or more sensors includes a carbon dioxide sensor.

20. The apparatus according to claim 19, wherein the transmitting member is configured to transmit carbon dioxide data at a given point of time if the carbon dioxide data is qualified by the controller.

21. The apparatus according to claim 19, wherein the identification mechanism is configured to include output data if carbon dioxide data at a given point of time is qualified by the controller or the external unit.

22. The apparatus according to claim 20, wherein the transmitting member is configured to transmit motion data, audio data, humidity data, temperature data and carbon dioxide data at a given point of time if the motion data, the audio data, the humidity data, the temperature data, and the carbon dioxide data are qualified by the controller.

23. The apparatus according to claim 1, wherein the one or more water consumption locations in a bathroom comprises a space defined by inside of walls of the bathroom, excluding locations within or outside the walls of the bathroom.

24. The apparatus according to claim 1, wherein the housing further comprises a commands receiving member configured to receive commands based on the qualified data at a given point of time from the external unit by low power signals, and the controller is configured to integrate the commands.

25. The apparatus according to claim 1, wherein the apparatus comprises a bracket with a magnetic member, and the housing comprises a hall sensor to detect the magnetic member upon placing the housing in the bracket whereby the apparatus is activated.

26. The apparatus according to claim 1, wherein the housing comprises:
a front cover including a transparent centrally arranged portion facing a front of said cover that allows visibility to a display positioned below the transparent centrally arranged portion, and a portion with a thinner wall thickness that allows functioning of the motion sensor located below a level of a surface of the housing;
a back cover connectable and sealable with the front cover, the back cover encasing a printing board, the printing board including the display operable to visualize the unique matrix barcode, the one or more sensors, the controller, and the transmitting member; and
a back plate connectable and sealable with the back cover, the back plate and the back cover encasing a power supply member for operating the apparatus.

27. The apparatus according to claim 1, wherein the output data includes one or more of water-saving tips, shower statistics, water tap statistics, toilet usage statistics, running water statistics and running water alerts.

28. A system for reducing or controlling water consumption in bathrooms, the system comprising:
an apparatus according to claim 1;
an external unit configured to receive qualified data from the apparatus at a given point of time, optionally via mobile device, by low power signals, and being configured to process data based on the qualified data and/or based on further qualification of data and configured to incorporate output data in a unique identification mechanism being readable by a mobile device on the apparatus for reducing or controlling water consumption in a bathroom;
optionally a commands receiving member in the apparatus configured to receive commands based on the qualified data at a given point of time from the external unit by low power signals, and the controller being configured to integrate the commands; and
a mobile device operable to read the unique identification mechanism.

29. The system according to claim 28, further including one or more external sensors communicably coupled to the apparatus, optionally by Bluetooth, and further including a sensor receiving member in the apparatus configured to receive data from the one or more external sensors, and the controller being configured to integrate the data.

30. The system according to claim 29, wherein the one or more external sensors include one or more external temperature sensors mounted to a water inlet of a local installation in the bathroom, including one or more inlets of a toilet, a water tap or a shower, and the one or more external temperature sensors being configured to detect a temperature difference of water in the inlet compared to ambient temperature over time, whereby running water may be detected.

31. The system according to claim 29, wherein the one or more external sensors includes an external temperature sensor mounted to a water inlet of a first local installation in the bathroom in combination with an external audio sensor in proximity to a water inlet of a second local installation in the bathroom and/or an external temperature sensor mounted to the water inlet of a second local installation in the bathroom whereby coordinated usage of both installations may be detected.

32. The system according to claim 28, further including an alert for running water to the mobile device and/or to the external unit.

33. The system according to claim 28, wherein the apparatus is included in a plurality of bathrooms, and the external unit is configured to process metadata based on qualified data from a plurality of bathrooms, optionally including said metadata in the unique identification mechanism and/or a central dashboard.

34. The system according to claim 29, wherein the one or more external sensors is located within a space defined by inside of walls of the bathroom, excluding locations within or outside the walls of the bathroom.

35. A method of reducing or controlling water consumption in bathrooms, the method comprising the steps of:
providing an apparatus being configured to receive data from one or more water consumption locations in a bathroom, the apparatus comprising a housing including one or more sensors, a controller, a transmitting member, and a unique identification mechanism;
detecting a movement in a threshold distance from the apparatus in a bathroom as a first indication of activity in the bathroom by a motion sensor, detecting a sound of running water from one or more water consumption locations in a bathroom as a second indication of activity in the bathroom by an audio sensor, optionally detecting the ambient humidity in a bathroom as a third indication of activity in the bathroom by a humidity sensor and optionally detecting ambient temperature by an ambient temperature sensor;
qualifying at least a part of the data received from the one or more sensors by an algorithm to obtain qualified data;
transmitting the qualified data at a given point of time to an external unit, optionally via a mobile device, by low power signals; and
subjecting output data based on the qualified data for reducing or controlling water consumption in the bathroom to a unique identification mechanism being readable or scannable by a mobile device.

36. The method according to claim 35, wherein the audio sensor is protected from water droplets in the apparatus by a trap that serves to avoid water interference without substantially reducing audio signals, the trap comprising a U-shaped air channel starting from the face of a bottom portion of the apparatus and extending towards the audio sensor.

37. The method according to claim 35, wherein the humidity sensor is protected from water droplets in the apparatus by a trap that serves to avoid water interference without substantially reducing audio signals, the trap comprising a U-shaped air channel starting from the face of a bottom portion of the apparatus and extending towards the humidity sensor.

38. The method according to claim 35, wherein a bracket of the apparatus is fixed to the housing by a connecting member adjusted to secure that the audio sensor in the housing is tuned for optimized sound picture.

39. The method according to claim 35, wherein the method is applied in an apparatus and/or a system for reducing or controlling water consumption in bathrooms.
